(19) United States
(12) Patent Application Publication
(10) Pub. No.: US 2010/0292539 A1
(43) Pub. Date: Nov. 18, 2010

(54) Forward Scanning OCT Endoscope

(75) Inventors: Eva Lankenau, Rondeshagen (DE); Tim Bonin, Hamburg (DE); Gereon Huettmann, Luebeck (DE)

Correspondence Address: DIEDERIKS & WHITELAW, PLC, 13885 HEDGEWOOD DR., SUITE 317, WOODBRIDGE, VA, 22193, US

(73) Assignee: Medizinisches Laserzentrum Luebeck GmbH, Luebeck (DE)

(21) Appl. No.: 12/780,352

(22) Filed: May 14, 2010

(30) Foreign Application Priority Data
May 15, 2009 (DE) 10 2009 021 580.8

(51) Int. Cl. A61B 1/07

(52) U.S. Cl. 600/167

(57) ABSTRACT

An apparatus for optical coherence tomography has a broadband light source with a short coherence length, an optical fiber that guides the light from the light source to a focusing optics, and a graded-index optics arranged between the optical fiber and the focusing optics with two opposite parallel flat sides, that is contacted on its first flat side by the optical fiber forming an irradiation point guiding light to the graded-index optics and having a pitch of N/8, N being a natural number that cannot be divided by 4. A first structure for light reflection is arranged on the first flat side of the graded-index optics adjacent to the irradiation point, and a second structure for beam splitting is arranged on the second flat side of the graded-index optics. The focusing optics are designed for focusing the light transmitted by the second structure essentially at right angles to the flat sides of the graded-index optics.

### BACKGROUND OF THE INVENTION

The invention relates to an apparatus for Optical Coherence Tomography (OCT) using an applicator that is designed in the fashion of an endoscope for introduction into cavities, in particular body cavities of a living human being or animal.

The OCT method is presently state of the art in medical diagnostics. Here broadband light with a short coherence length from a suitable light source (e. g. from a superluminescent diode) is at first subdivided into a sample and a reference light beam, and then the sample beam is directed at the object to measured (e. g. living tissue such as epidermis, retina) and reflected back by it into different depths. In contrast, the reference light is in principle reflected from a reference mirror and guided such that it has passed approximately the same optical path length as the sample light when it is finally rejoined with the returning sample light in an evaluation unit and superposed thereon. The signal of interest (the depth-resolved distribution of the scattering strengths in the object) then results from the intensity of the interference light which is only produced if the light paths of the sample and reference beams do not differ by more than the coherence length of the light.

In the case of the time-dependent OCT (“time domain”, TD) the length of the reference branch of the interferometer is of variable design and is usually varied periodically by means of a so-called phase modulator. At defined times, the interference light can only originate from specific depths in the object. The realization and control of such phase modulators is technically complex and relatively expensive.

Simplified OCT apparatus that make do with a fixed reference branch length and thus without moving parts are known from WO 02/084263 A1 and from DE 43 09 056 A1.

The method of DE 43 09 056 A1 is usually referred to as a “spectral radar” or also “Fourier domain” (FD-OCT). Here light from a broadband light source is scattered in the sample in a plane at a distance z from a reference plane (z=0) and superposed with backscattered light from the reference plane. This results in constructive or destructive interference for any desired fixed distance z of the planes depending on which of the irradiated wavelengths λ are observed. The interference light is therefore split up spectrally and usually imaged onto a line of photo diodes or a similar device. This permits the distribution I(k), k=2π/λ, to be measured as a spatial distribution on the sensor line. A Fourier transformation of this distribution leads to the depth-dependent scattering potential S(z).

In the so-called Linear OCT (L-OCT) from WO 02/084263 A1, sample and reference lights are initially generated and guided as in the case of the spectral radar mentioned and finally superposed spatially as in the known double-slit experiment. The interference light forms a strip pattern on a line sensor (such as a CCD sensor), where each pixel of the sensor is associated with a different travel time of the light—and thus a different depth in the sample. Here the envelope of the intensity distribution along the sensor row contains the sample information.

Both methods are characterized in that the reference mirror of the interferometer can be positioned close to the sample. Sample and reference lights can be guided simultaneously in the same optical fiber over long distances.

This has the great advantage if the sample light is to be radiated onto the object to be measured (mostly a patient) using a moving applicator—possibly hand-held—and guided back from there into a stationary evaluation unit. Every movement and/or twisting of the fiber, but also temperature, variations or mechanical stress, influence the travel time of the sample light and lead to interference signals relative to a reference branch that is not exposed to these factors when the sample and reference lights are superposed. Guiding both lights in the same fiber eliminates this problem.

However this means that the reference mirror, too, has to be accommodated in the applicator.

Especially in the case of an applicator that is to be used like an endoscope it is of course specified that the applicator has to have a shape that is as narrow, flexible and tube-like as possible.

A suggestion for the realization can be gathered from WO 2009/140617 A2 or also U.S. 2008/228033 A1. In the printed publication that was the last one to be mentioned it is provided to design the distal end of the endoscope in such a way that light leaving the optical fiber is at first focused by means of a graded-index lens (GRIN) into the object material (thus organic tissue) situated to the side of the end of the endoscope. In the process a beam splitter is arranged behind the GRIN lens that directs part of the beam into the tissue through an exit window provided to the side of the end of the endoscope and guides the light returning from there through the GRIN lens back into the fiber. The second part of the beam is for example allowed to pass in the forward direction of the endoscope to a mirrored surface that serves as reference mirror in the meaning of the OCT. As an alternative it is also suggested to use the lateral exit window itself both as beam splitter and also as reference mirror, for example by providing a part-reflective coating on the exit window.

The suggestion of U.S. 2008/228033 A1 can be implemented well in a compact and above all slim design. However it has the disadvantage that the OCT measurement can only succeed to the side in the tissue immediately neighboring onto the endoscope.

The possibility would however be desirable for OCT measurements in the forward direction of the endoscope which at the same time can mean that the object to be investigated has a spacing of a few millimeters from the exit window of the distal endoscope end. Although it is unproblematic to design a sufficiently large focal length of the focusing optics, but providing the beam splitter and the reference mirror in or at the forwardly facing exit window is not a favorable measure since sample and reference branch lengths of the interferometer will then differ by too much. Rather a light path of appropriate length has to be provided for the reference light, in which case the reference mirror that is required specifically cannot be arranged in the forward direction of the endoscope.

The suggestion is obvious and can also be gathered from the prior art to simply rotate the light-guiding arrangement of U.S. 2008/228033 A1 behind the focusing optics through 90°, i. e. to deflect the reference light at right angles to the longitudinal axis of the endoscope and to guide it to a reference mirror that is arranged laterally. If however this laterally facing reference branch requires a length of a few millimeters either the setup will become very complicated or the distal end of the endoscope will not become very narrow.

In endoscopes it is common to use GRIN lenses for focusing purposes. They typically have an index of refraction profile that extends radially and is parabolic, the consequence of which is that light beams that are coupled in take a sinusoidal course inside the GRIN optics. In this respect the GRIN optics has an intrinsic period length, also called pitch. A lens having the length of 1 pitch has the attribute that a light beam injected on the one side in any arbitrary direction leaves the optics on the other side in precisely the same direction. The light of a point source on the entry surface is focused onto a point of the exit surface that is precisely opposite the point source.

A GRIN optics having the length of ¼ pitch however collimates the light of this point source.

These facts result in the applicability of GRIN optics having a length n/4 (n—odd integer) pitch as retroreflectors, as can be gathered e. g. in U.S. Pat. No. 4,789,219.

Use of GRIN optics as retroreflectors has already been suggested in EP 1 647 798 A1 also for interferometric measurements. This printed publication is about an interferometric method for determining the position of surfaces whose orientation is not known in advance or even is variable. The sample light is therefore not irradiated at right angles onto the surface of the object to be measured but at a tilting angle. As a consequence the reflected sample light does not return to the injection direction immediately but a downstream retroreflector is required for this to be achieved. The idea of EP 1 647 798 A1 now consists among others in using a GRIN optics with ¼ pitch both for collimating the light injected from a point source (e. g. monomode glass fiber) and also as retroreflector in the sample branch. In all designs of the invention, the reference branch of the interferometer is oriented at right angles to the injection direction (note there in particular FIGS. 4A and B that illustrate different side views of the same apparatus).

### SUMMARY OF THE INVENTION

The object of the invention is to suggest an applicator for an OCT system that has a particularly compact and narrow design and is therefore suitable for using like an endoscope, permitting the OCT measurement in the advancing direction (subsequently also directed in the forward direction).

The object is achieved by an apparatus having the features of the main claim. The sub-claims specify advantageous developments.

The inventive applicator comprises an optical fiber that according to the prior art is suited for guiding the OCT light in a flexible tube that is likewise known per se. According to the invention, the distal end of the tube is provided with an arrangement in the following order: a GRIN device is arranged behind the exit end of the optical fiber, behind this a focusing optics and possibly an exit window in the advancing direction, the focusing optics being arranged for focusing the light in the advancing direction of the applicator.

According to the invention, the GRIN device comprises a GRIN optics known per se with a proximal (fiber-side) and a distal (opposite the fiber) flat side. A mirror is arranged on or immediately in front of the proximal flat side in the vicinity of the fiber exit. A beam splitter is arranged on or immediately behind the distal flat side. To this end, it is particularly preferred that the flat sides of the GRIN optics are provided with functional layers, in particular with a highly reflective layer on the proximal flat side and a part-reflective layer on the distal flat side. The precise design of the mirror and the beam splitter does not matter. It is however essential that the mirror planes of light reflection through mirror and beam splitter coincide with the flat sides of the GRIN optics.

According to the invention, the length of the GRIN optics essentially represents the length of the reference branch of the OCT system. Since it is now oriented in the direction of the longitudinal axis of the tube, i. e. in the advancing direction, relative long lengths are possible in conjunction with a narrow design of the applicator.

The precise length of the GRIN optics can be chosen by the manufacturer, and care always has to be taken that the travel times of the light in the sample and the reference branches in the interferometer have to be essentially identical for an OCT measurement to be carried out. For both light paths from the beam splitter to the reference mirror or the sample it has to hold that the ratios of the path lengths to the respective group velocities of the light (if necessary summed up over part path lengths with different group velocities) essentially coincide.

According to the invention, the index profile of the GRIN optics now has to be set in such a way that the selected length of the GRIN optics forms a pitch length of N/8, N having to be selected from the set of natural numbers than cannot be divided by 4. To illustrate this: N=1, 2, 3, 5, 6, 7, 9, . . . are possible.

The GRIN device of this invention therefore comprises—preferably in a structural unit—the reference mirror of the interferometer, a GRIN optics with N/8 pitch (N see above) at any selected wavelength, and the beam splitter of the interferometer. Only light that has passed the beam splitter is focused by the focusing optics into the tissue and leaves the applicator in the forward direction through the exit end. The focusing optics is there alone in the sample branch of the interferometer. In particular the GRIN optics of the inventive GRIN device is not part of the focusing optics.

The focusing optics can nevertheless of course also be designed as a GRIN lens but this is not important for the invention. Any other design for focusing is also suitable, such as a plano-convex lens.

### BRIEF DESCRIPTION OF THE DRAWINGS

The invention is explained in more detail below with reference to two figures, in which:

FIG. 1 shows the beam path in an inventive applicator, where the optical fiber radiates in a centered fashion into the GRIN device and N=5;

FIG. 2 shows the beam path in an inventive applicator, where the optical fiber does not radiate in a centered fashion into the GRIN device and N=2.

### DETAILED DESCRIPTION OF THE INVENTION

The invention in principle takes advantage of the image-conducting attributes of GRIN optics so as to realize the reference branch using simple means. Here in principle two variations are possible, depending on whether an axially symmetric light-guiding arrangement is to be realized or not.

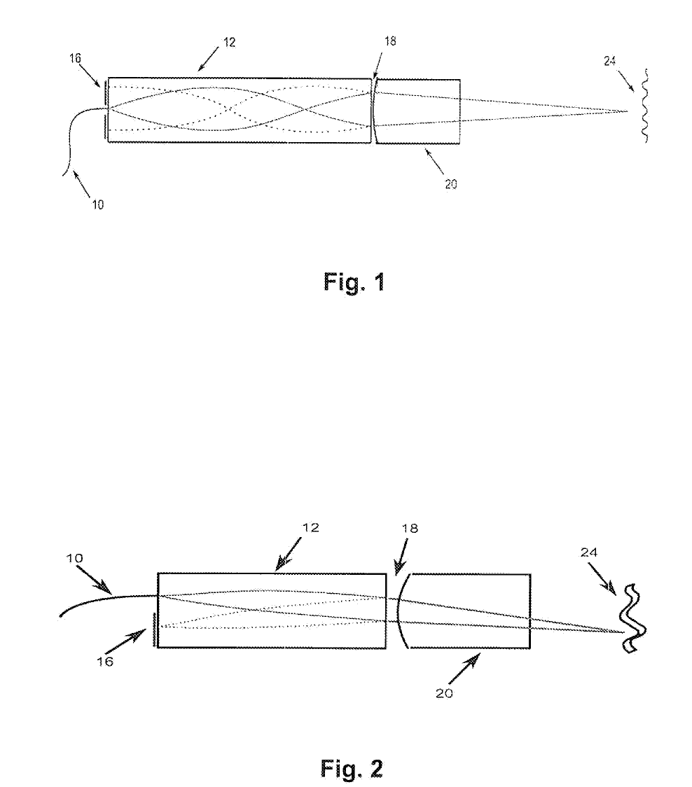

FIG. 1 represents the symmetric case. Here N has to be an odd number, for example N is set=5. The optical fiber 10 is connected (usually glued) to the inventive GRIN device 12 in such a way that the light leaves the fiber precisely at the center of the proximal flat side along the axis of symmetry of the GRIN device. The proximal flat side is completely mirrored with the exception of the surroundings of the fiber end, i. e. a highly reflective coating 16 that is interrupted at the center is applied to the proximal flat side. The continuous lines indicate the beam path of the light cone that starts from the fiber end and at first leads to the distal flat side of the GRIN device where the light is split by a partly reflective coating 18. The transmitted light component is focused by a focusing optics 20 (here as an example a plano-convex lens) in the forward direction into the object 24 to be measured and scattered back from there. After this the sample light or scattered light takes the same path back to the optical fiber 10. The light component reflected from the beam splitter—the reference light—again traverses the GRIN device 12 (dotted line). After having reached the mirror surface 16, due to its setup it has covered an additional path corresponding to the focal length of the focusing optics 20 and in total has passed through a GRIN optics with 2*N/8=N/4 pitch and N being odd. As a consequence it is mirrored back precisely in itself, in the process again reaching the part reflective layer 18, and from there it is focused again proportionately to the fiber exit end at the center of the proximal flat side, together with the sample light returning from the measurement object 24. A small proportion of the reference light is lost at the interrupted layer 16 during mirroring since it enters into the fiber 10, which does not affect the evaluation. A larger share of the reference light is lost during the second reflection at the layer 18 since it leaves the applicator in the forward direction.

FIG. 2 shows the case where the beam is guided asymmetrically, where N is to be even but not a multiple of 4. As an example N has been set to 2. The optical fiber 10 is arranged here outside the symmetry axis of the GRIN optics. The inventive GRIN optics collimates the light from the fiber 10. The collimated light always leaves the distal flat side of the GRIN device 12 at an angle different from zero against the symmetry axis of the GRIN optics. Here, too, a partly reflective layer 18 is arranged on the distal flat side. The light component guided back from the beam splitter 18 into the GRIN optics (here, too, the reference light) is now focused onto a point on the proximal side of the GRIN optics. This focal point lies in the proximal flat side, adjacent to the fiber exit, but opposite it centrosymmetrically. It is therefore sufficient to apply a highly reflective layer 16 only to this relatively small surface in the vicinity of the fiber exit. Again the GRIN optics functions as a retroreflector, and after being reflected again at the beam splitter 18 it returns again into the fiber 10. What has been said in FIG. 1 also holds for the sample light.

At this juncture it is easy to explain why N=4, 8, 12, . . . are not suited, since after traversing the GRIN optics twice the reference light beam would then be reflected back directly into the fiber end and would be lost for evaluation in the interferometer.

In the two figures described above no exit window is illustrated. To the person skilled in the art it is obvious that it can coincide e. g. with the flat side of the plano-convex lens or also be a separate component. The design of the exit window is of no importance for the invention.

An advantageous design of the partly reflective layer (beam splitter 18) lies in coating the GRIN device with a dielectric material or with a thin metal film. For example a thin gold film is suited having a thickness of a few nanometers, preferably less than 20 nm.

It is also advantageous to split the beam geometrically. To this end, the distal flat side of the GRIN device is mirrored completely (such as metalized, e.g. with gold) except for a central circular aperture that is smaller than the beam diameter. Thus the outer area of the mode field is reflected, creating a geometric beam-splitting arrangement.

1. The apparatus for optical coherence tomography comprising:
a broadband light source with a short coherence length;
a focusing optics;
an optical fiber that guides light from the light source to the focusing optics;
a graded-index optics arranged between the optical fiber and the focusing optics with two opposite parallel flat sides, that is contacted on its first flat side by the optical fiber forming an irradiation point guiding light to the graded-index optics and having a pitch of N/8, N being a natural number that cannot be divided by 4;
a first means for light reflection, arranged on the first flat side of the graded-index optics adjacent to the irradiation point; and
a second means for beam splitting arranged on the second flat side of the graded-index optics, wherein the focusing optics focuses the light transmitted by the second means essentially at right angles to the flat sides of the graded-index optics.

2. An apparatus according to claim 1, wherein the irradiation point is at the center of the first flat side of the graded-index optics, the first means covers the first flat side over the whole surface with the exception of the irradiation point and N is an odd natural number.

3. The apparatus according to claim 1, wherein the irradiation point is outside the center of the first flat side of the graded-index optics, the first means covers that point of the first flat side that results from point reflection of the irradiation point at the center of the first flat side, and N is an even natural number that cannot be divided by 4.

4. The apparatus according to claim 1, wherein the first means is formed on the first flat side of the graded-index optics as a highly reflective first coating.

5. The apparatus according to claim 4, wherein the first coating is an opaque metal film.

6. The apparatus according to claim 4, wherein the second means is provided on the second flat side of the graded-index optics as a partly reflective second coating.

7. The apparatus according to claim 6, wherein the second coating is a gold film having a thickness of less than 20 nm.

8. The apparatus according to claim 6, wherein the second coating, with the exception of an area that remains free at the center of the flat side, is designed over the whole surface and is highly reflective, while the area that remains free is smaller than a diameter of the beam.
